(19) United States
(12) Patent Application Publication
(10) Pub. No.: US 2025/0174354 A1
(43) Pub. Date: May 29, 2025

(54) WORKFLOW ENHANCEMENT IN SCREENING OF OPHTHALMIC DISEASES THROUGH AUTOMATED ANALYSIS OF DIGITAL IMAGES ENABLED THROUGH MACHINE LEARNING

(71) Applicant: Verily Life Sciences LLC, Dallas, TX (US)

(72) Inventor: Sam KAVUSI, Menlo Park, CA (US)

(21) Appl. No.: 18/841,349

(22) PCT Filed: Mar. 8, 2023
(86) PCT No.: PCT/US2023/063983

§ 371 Date: Aug. 23, 2024

Related U.S. Application Data
(60) Provisional application No. 63/269,239, filed on Mar. 11, 2022.

(51) Int. Cl. G16H 50/20; G06T 7/00

(52) U.S. Cl. CPC G16H 50/20; G06T 7/0012; G06T 2207/20084; G06T 2207/30041; G06T 2207/30168

(57) ABSTRACT

Introduced here are approaches to assessing digital images generated during image capture sessions using a machine learning model so as to stratify patients for examination. By applying the machine learning model to the digital images, the patients that are most in need of further examination can be identified to graders. For example, outputs produced by the diagnostic model may trigger the generation and transmission of notifications for patients that are deemed to warrant further examination.

### CROSS-REFERENCE TO RELATED APPLICATIONS

This application claims priority to U.S. Provisional Application No. 63/269,239, titled “Workflow Enhancement in Screening of Ophthalmic Diseases Enabled Through Machine Learning” and filed on Mar. 11, 2022, which is incorporated herein by reference in its entirety.

### TECHNICAL FIELD

Various embodiments concern computer programs and associated computer-implemented techniques for stratifying patients for examination by healthcare professionals based on analysis of the pathological features in digital images.

### BACKGROUND

Fundus photography involves capturing a digital image of the fundus to document the retina, which is the neurosensory tissue in the eye that translates optical images into the electrical impulses that can be understood by the brain. The fundus can include the retina, optic disc, macula, fovea, and posterior pole.

Fundus cameras (also called “retinal cameras”) are designed to provide an upright, magnified view of the fundus. Generally, a subject (also called a “patient”) will sit at the retinal camera with her chin set within a chin rest and her forehead pressed against a bar. An operator may be responsible for visually aligning the retinal camera and then pressing a shutter release that causes a digital image of the retina to be generated. Healthcare professionals, such as optometrists, ophthalmologists, and orthoptists, may use the digital images generated by retinal cameras to detect and/or monitor ocular diseases (also called “eye diseases” or “eye conditions”). For example, the digital images could be used to document indicators of diabetic retinopathy, age-related macular degeneration (AMD), glaucoma, and the like.

For many reasons-including costs and practicality-telemedicine is becoming an increasingly attractive option for examining digital images for the purpose of diagnosing ocular diseases. The term “telemedicine” refers to the practice of medicine that involves utilizing technology to deliver care at a distance. Generally, a healthcare professional in one location uses a telecommunications infrastructure (e.g., the Internet) to deliver care to a patient at another location.

### BRIEF DESCRIPTION OF THE DRAWINGS

FIG. 1 includes a diagrammatic illustration of a conventional screening workflow for an ocular disease via telemedicine.

FIG. 2 includes a diagrammatic illustration of a screening workflow in which graders are notified of digital images requiring examination.

FIG. 3 includes a diagrammatic illustration of a screening workflow in which a diagnostic model is applied to digital images by a diagnostic platform so that the patients requiring further examination can be identified to graders.

FIG. 4 illustrates a network environment that includes a diagnostic platform.

FIG. 5 illustrates an example of a computing device that includes a diagnostic platform able to determine whether further examination of a digital image associated with a patient is warranted.

FIG. 6 depicts an example of a communication environment that includes a diagnostic platform configured to acquire data from one or more sources.

FIG. 7 includes a flow diagram of a process for determining whether a digital image associated with a patient warrants further examination.

FIG. 8 includes a flow diagram of a process for determining whether digital images associated with different patients warrant further examination.

FIG. 9 includes a flow diagram of a process for handling digital images that are difficult to grade.

FIG. 10 depicts a flow diagram of a process for establishing whether expedited examination of a digital image associated with a patient is warranted.

FIG. 11 depicts a flow diagram of a process for stratifying patients whose retinas have been imaged for further examination.

FIG. 12 is a block diagram illustrating an example of a processing system that can implement at least some of the operations described herein.

Various features of the technology described herein will become more apparent to those skilled in the art from a study of the Detailed Description in conjunction with the drawings. Various embodiments are shown in the drawings for the purpose of illustration. However, those skilled in the art will recognize that alternative embodiments may be employed without departing from the principles of the technology. Accordingly, although specific embodiments are shown in the drawings, the technology is amenable to various modifications.

### DETAILED DESCRIPTION

In ophthalmology, telemedicine usually involves the remote gathering of digital images with subsequent analysis by healthcare professionals to identify those patients who have ocular diseases that warrant further consideration. Screening programs exist for several ocular diseases, including diabetic retinopathy, AMD, and glaucoma.

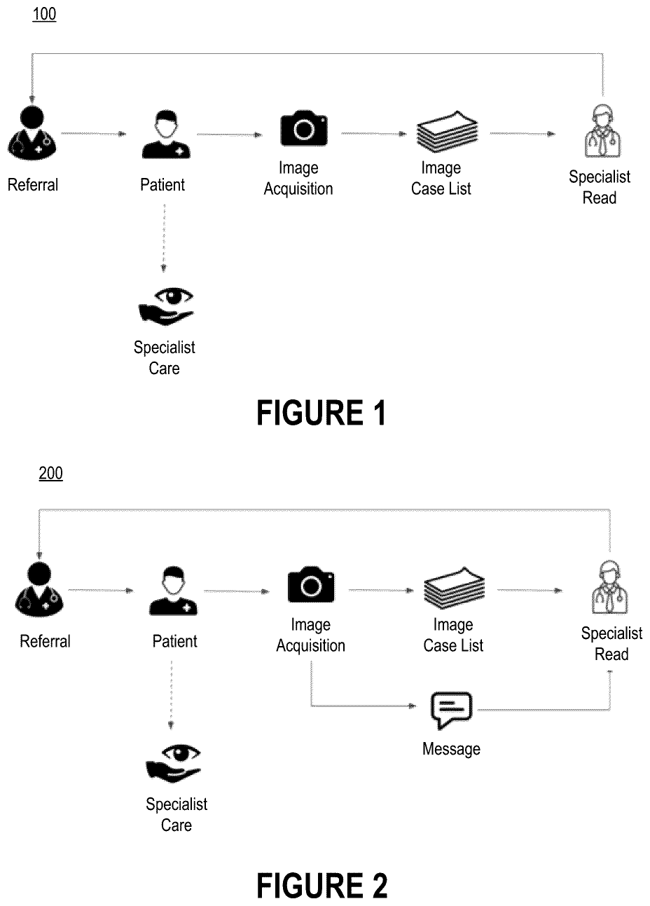

FIG. 1 includes a diagrammatic illustration of a conventional screening workflow 100 for an ocular disease via telemedicine. Initially, the patient may be referred for imaging by a first healthcare professional that has determined the patient may have the ocular disease. This determination may be based on symptoms reported by the patient, or this determination may be based on other indicators discovered by the first healthcare professional. The first healthcare professional could be the primary care physician of the patient.

After acquisition of one or more digital images, the typical latency of a read (also called an “examination”) by a second healthcare professional via telemedicine is normally between several minutes and several days. Generally, a small percentage (e.g., between one and five percent) of patients are then referred for treatment based on the reads performed by the second healthcare professional. This treatment could be provided by the second healthcare professional or another healthcare professional (e.g., the first healthcare professional or a third healthcare professional).

Generally, the second healthcare professional is an ophthalmic specialist who is responsible for examining the digital images to determine whether treatment is warranted. In addition to “ophthalmic specialist,” the second healthcare professional may also be referred to as a “grader.” The operation of telemedicine normally involves multiple graders, each of whom is able to access a record of the patients (and thus, digital images) awaiting examination and then providing the read. The actual read may only take several seconds or minutes to perform. Most of the latency in due to the availability of graders that may wait until breaks (e.g., lunch) or the conclusion of the workday to examine digital images. Said another way, although a grader may be able to determine whether examination is warranted relatively quickly, the grader may be delayed in actually performing that examination. The number of patients that can be screened via telemedicine can vary significantly depending on the number of active sites where imaging can be performed and the number of graders, among other factors.

The speed of referral for treatment can make a major difference in the speed and adherence of treatment, and therefore in saving vision. One approach involves triggering a quick read by a grader. FIG. 2 includes a diagrammatic illustration of a screening workflow 200 in which graders are notified of digital images requiring examination. Transmitting a message to a list of graders, so as to trigger a read of a digital image, can serve as an approach to quickening the pace at which decisions are made. This approach can reduce the time needed for a digital image to be graded below ten minutes, and therefore ensure that patients in near real time after the eye has been imaged. However, this approach lacks optimal efficiency and scalability.

Introduced here, therefore, are approaches to assessing digital images generated during image capture sessions using a machine learning model (also called a “diagnostic model”) so as to stratify patients for examination. Appropriate stratification improves outcomes-if patients with more severe diseases are examined before patients with less severe diseases, better outcomes are generally achieved—and also improves the efficiency of examination and reduces consumption of necessary human resources and computational resources. Patients whose diseases have been misdiagnosed or appropriately-yet-belatedly diagnosed generally require that additional time be spent by healthcare professionals and additional resources (e.g., in terms of healthcare equipment, medications, etc.) be used by healthcare professionals in examining and treating those patients. Misdiagnoses and belated diagnoses also tend to require more computational resources, at a granular level (e.g., because diagnostic models may need to be applied to digital images generated over multiple image capture sessions) and at a holistic level (e.g., because more appointments need to be rescheduled, more digital images need to be captured, etc.). As noted above, the approaches introduced here allow for patients to be stratified in a more efficient (and scalable) manner that relies on automated analysis of digital images using one or more diagnostic models.

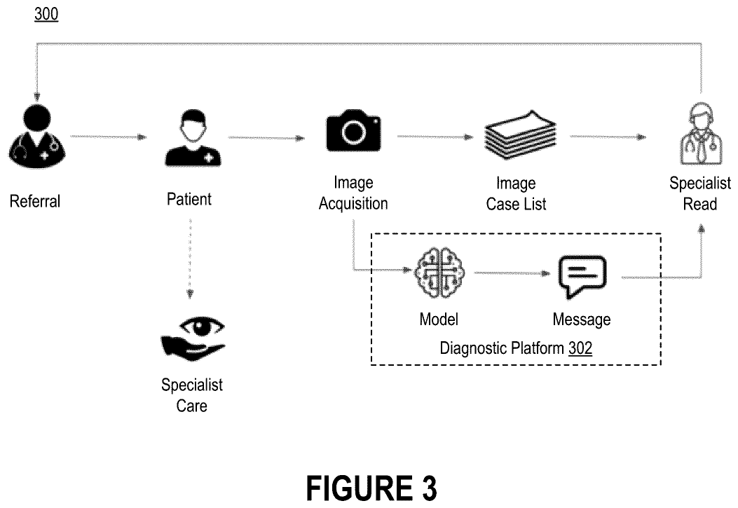

FIG. 3 includes a diagrammatic illustration of a screening workflow 300 in which a diagnostic model is applied to digital images by a diagnostic platform 302 so that the patients requiring further examination can be identified to graders. As further discussed below, the diagnostic model can be designed and then trained to produce outputs that are indicative of proposed diagnosis for an optical disease. Outputs produced by the diagnostic model can trigger the generation and transmission of notifications for patients that are deemed to warrant further examination.

Assume, for example, that the diagnostic platform 302 acquires a digital image that is generated for the purpose of remotely diagnosing the presence or severity of an ocular disease in a patient. In such a scenario, the diagnostic platform 302 can apply a diagnostic model to the digital image, so as to produce an output. The output may be indicative of or associated with a proposed next step diagnosis for the ocular disease. The proposed next step diagnosis can be a final diagnosis to be entered in the patient's medical record (e.g., electronic medical record), a prognosis of a disease progression, a companion diagnosis of the applicability of a treatment option, a severity grading of how severe the disease state is, or any combination thereof. The diagnostic platform 302 can then determine what action, if any, to take based on the output. For example, if the output is indicative of a positive diagnosis for the ocular disease, then a notification may be transmitted to a list of graders to prompt further examination of the given digital image. Said another way, the diagnostic platform 302 can transmit a notification to prompt a read by one of the graders included on the list. Conversely, if the output is indicative of a negative diagnosis for the ocular disease, then no further action may be taken by the diagnostic platform 302. Note that the term “positive diagnosis” may be used to refer to a scenario where a patient is diagnosed as having the ocular disease, while the team “negative diagnosis” may be used to refer to a scenario where a patient is diagnosed as not having the ocular disease.

In various embodiments, the “diagnostic model” used is a patient screening model capable of classifying and/or triaging medical images for further examination. In these embodiments, the diagnostic model can also be referred to as a screening model. Note that while the approaches may be described in the context of these diagnostic models that are used specifically for screening, in other embodiments, diagnostic models beyond screening are also contemplated, such as for providing a diagnostic in lieu of a grader, for grading or estimating a severity of a disease, for prognosis on estimated progression of the disease, for determining or proposing efficacy of a treatment option (e.g., companion diagnostic), etc.

Generally, the digital image is one of multiple that the diagnostic platform 302 is tasked with examining. For example, the diagnostic platform 302 may be responsible for examining some or all of the digital images generated by retinal cameras located in different settings (e.g., different healthcare facilities). Applying the diagnostic model to these digital images may permit the diagnostic platform 302 to stratify the corresponding patients for examination. For example, the diagnostic platform 302 may prompt quicker review of digital images that are determined to have evidence of an ocular disease, or the diagnostic platform 302 may prompt quicker review of digital images that are determined to have evidence of greater severity of an ocular disease.

At a high level, this approach to expediting examination not only results in an optimized workflow, but also improved clinical management as those patients in need of treatment can be examined more quickly. When implemented, this approach leads to several benefits. First, the relatively small percentage of patients that require referral are able to receive results in a short amount of time (e.g., several minutes) while the remaining patients can simply receive results in accordance with a typical timeframe (e.g., several minutes to several days). Second, diagnostic accuracy in the aggregate can be improved as each grader is able to confirm or reject the diagnosis proposed by the diagnostic model. Third, operational cost is relatively low since the computational resources needed to facilitate notification of the graders (and examination by one of those graders) may only be expended when there is a high likelihood of a positive diagnosis. Fourth, patients whose eyes are difficult to image can be handled efficiently. As further discussed below, a quality check may be implemented by the diagnostic platform 302 to ensure that graders are not tasked with examining digital images of poor quality.

Machine learning—and more specifically, deep learning—has permitted the learning and detecting of digital features (or simply “features”) that are representative of referable pathological conditions. However, diagnostic models tend to have limited coverage, as each diagnostic model will only detect features indicative of the pathological condition that it was trained to diagnose. For this reason, the diagnostic platform may apply multiple diagnostic models to offer broader coverage as further discussed below.

Note that while the approaches may be described in the context of diagnostic models designed and developed for deep learning, other implementations are possible. Instead of, or in addition to, deep learning models, the diagnostic platform may (i) perform computer vision-based feature detection or (ii) implement algorithmic decision making based on analysis of the content of a digital image, its metadata, related patient information, or any combination thereof. The diagnostic platform may configure how it considers digital images provided as input based on the computational resources available to it, the desired speed at which the digital images are to be reviewed, and the like.

Embodiments may be described with reference to particular types of ocular diseases for the purpose of illustration. For example, embodiments may be described in the context of stratifying patients who are determined to have diabetic retinopathy. However, those skilled in the art will recognize that these features are similarly applicable to other types of ocular diseases. In fact, the approach described herein could be applied to stratifying patients for examination regardless of the pathological condition. Thus, the diagnostic platform could employ the approach described herein to screen and rank patients with nearly any type of disease for which presence or severity can be established by applying a diagnostic model to a digital image of a corresponding anatomical region.

While embodiments may be described in the context of computer-executable instructions, aspects of the technology can be implemented via hardware, firmware, or software. As an example, a set of algorithms indicative of a diagnostic model designed to detect features that are diagnostically relevant may be executed by a diagnostic platform. The diagnostic platform could be embodied as a software program that offers support for reviewing digital images, rendering diagnoses, and cataloging treatments. In particular, the diagnostic platform may prompt a processor to execute instructions for acquiring a digital image generated by a retinal camera), applying the diagnostic model to the digital image to detect diagnostically relevant digital features, producing a proposed diagnosis for an ocular disease, and then determining whether to notify at least one grader to examine the digital image based on the proposed diagnoses.

### Terminology

References in this description to “an embodiment” or “one embodiment” means that the particular feature, function, structure, or characteristic being described is included in at least one embodiment. Occurrences of such phrases do not necessarily refer to the same embodiment, nor are they necessarily referring to alternative embodiments that are mutually exclusive of one another.

Unless the context clearly requires otherwise, the words “comprise” and “comprising” are to be construed in an inclusive sense rather than an exclusive or exhaustive sense (i.e., in the sense of “including but not limited to”). The term “based on” is also to be construed in an inclusive sense rather than an exclusive or exhaustive sense. Thus, unless otherwise noted, the term “based on” is intended to mean “based at least in part on.”

The terms “connected,” “coupled,” or any variant thereof is intended to include any connection or coupling between two or more elements, either direct or indirect. The connection/coupling can be physical, logical, or a combination thereof. For example, objects may be electrically or communicatively coupled to one another despite not sharing a physical connection.

The term “module” refers broadly to software components, firmware components, and/or hardware components. Modules are typically functional components that generate one or more outputs based on specified one or more inputs. A computer program may include one or more modules. Thus, a computer program may include multiple modules responsible for completing different tasks or a single module responsible for completing all tasks.

When used in reference to a list of multiple items, the word “or” is intended to cover all of the following interpretations: any of the items in the list, all of the items in the list, and any combination of items in the list.

The sequences of steps performed in any of the processes described here are exemplary. However, unless contrary to physical possibility, the steps may be performed in various sequences and combinations. For example, steps could be added to, or removed from, the processes described herein. Similarly, steps could be replaced or reordered. Thus, descriptions of any processes are intended to be open-ended.

### Overview of Diagnostic Platform

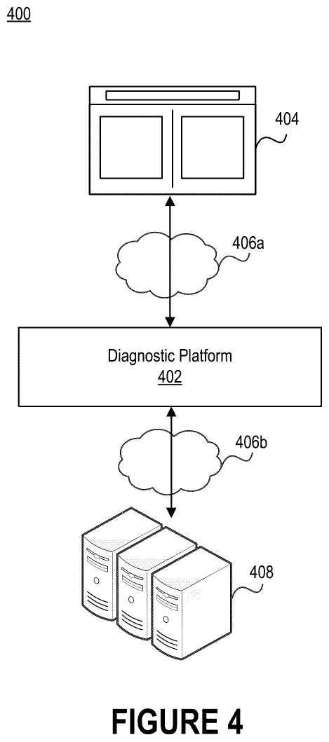

FIG. 4 illustrates a network environment 400 that includes a diagnostic platform 402. Individuals can interact with the diagnostic platform 402 via an interface 404. For example, healthcare professionals may access the interface 404 to review the digital images generated by an imaging device, such as a retinal camera, a mobile phone, or a digital camera (e.g., a digital single-lens reflex (DSLR) camera or a mirrorless camera), in order to diagnose the patients whose bodies are captured in those digital images. Moreover, healthcare professionals may access the interface 404 to review the outputs produced by diagnostic models that have been applied to those digital images.

Diagnostic models may be applied to digital images generated during a diagnostic session (also called an “image capture session”) in order to identify the regions of pixels that are diagnostically relevant. When applied to a digital image, a diagnostic model may produce an output that is indicative of the health state of a corresponding patient. Some diagnostic models are designed to produce a proposed diagnosis that indicates whether the patient has a pathological condition. Other diagnostic models are designed to specify a proposed classification that indicates severity of a pathological condition. For example, a diagnostic model may indicate whether the patient has mild, moderate, or severe diabetic retinopathy upon being applied to a digital image of the patient's eye. Diagnostic models may also be designed to produce visualization components (or simply “visualizations”) that are intended to help healthcare professionals render diagnoses. The term “health state” can refer to the physical health of the patient with respect to a given pathological condition. For example, a diagnostic platform could be designed to identify digital features that are known to be indicative of ocular diseases such as diabetic retinopathy, glaucoma, and the like.

As shown in FIG. 4, the diagnostic platform 402 may reside in a network environment 400. Thus, the diagnostic platform 402 may be connected to one or more networks 406a-b. The networks 406a-b can include personal area networks (PANs), local area networks (LANs), wide area networks (WANs), metropolitan area networks (MANs), cellular networks, the Internet, etc. Additionally or alternatively, the diagnostic platform 402 can be communicatively coupled to one or more computing devices over a short-range wireless connectivity technology, such as Bluetooth® or Near Field Communication (NFC).

The interface 404 is preferably accessible via a web browser, desktop application, mobile application, or over-the-top (OTT) application. Accordingly, the interface 404 may be viewed on a personal computer, tablet computer, mobile workstation, mobile phone, wearable electronic device (e.g., a watch or fitness accessory), a virtual/augmented reality system (e.g., a head-mounted display), or another network-connected electronic device (e.g., a television or home assistant device).

Some embodiments of the diagnostic platform 402 are hosted locally. That is, the diagnostic platform 402 may reside on the computing device used to access the interface 404. For instance, the diagnostic platform 402 may be embodied as a mobile application executing on a mobile phone or a desktop application executing on a mobile workstation. Other embodiments of the diagnostic platform 402 are executed by a cloud computing service operated by, for example, Amazon Web Services®, Google Cloud Platform™, or Microsoft Azure®. In such embodiments, the diagnostic platform 402 may reside on a network-accessible server system 408 comprised of one or more computer servers. These computer servers can include digital images generated by imaging devices, patient information (e.g., age, sex, health diagnoses, etc.), imaging device information (e.g., resolution, expected file size, etc.), diagnostic models, and other assets. Those skilled in the art will recognize that this information could also be distributed among a network-accessible server system and one or more computing devices.

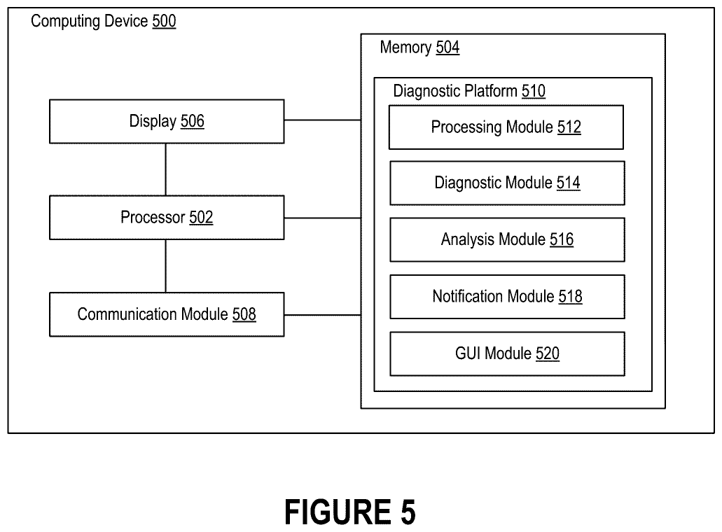

FIG. 5 illustrates an example of a computing device 500 that includes a diagnostic platform 510 able to determine whether further examination of a digital image associated with a patient is warranted. Such action enables the diagnostic platform 510 to notify graders when further examination is needed, so as to ensure that the examination is performed promptly. For example, if the diagnostic platform 510 discovers that a given patient likely has a given pathological condition based on an output produced by a diagnostic model, then the diagnostic platform 510 may notify multiple graders that confirmation is needed. As further discussed below, the multiple graders can be notified in such a manner that patients are considered in order of urgency.

The computing device 500 can include a processor 502, memory 504, display 506, and communication module 508. Each of these components is discussed in greater detail below. Those skilled in the art will recognize that other components may also be present depending on the nature of the computing device 500.

The processor 502 can have generic characteristics similar to general-purpose processors, or the processor 502 may be an application-specific integrated circuit (ASIC) that provides control functions to the computing device 500. As shown in FIG. 5, the processor 502 can be coupled to all components of the computing device 500, either directly or indirectly, for communication purposes.

The memory 504 may be comprised of any suitable type of storage medium, such as a static random-access memory (SRAM), dynamic random-access memory (DRAM), electrically erasable programmable read-only memory (EEPROM), flash memory, or registers. In addition to storing instructions that can be executed by the processor 502, the memory 504 can also store data generated by the processor 502 (e.g., when executing the modules of the diagnostic platform 510). Note that the memory 504 is merely an abstract representation of a storage environment. The memory 504 could be comprised of actual integrated circuits (also called “chips”).

The display 506 can be any mechanism that is operable to visually convey information to a user of the computing device 500. For example, the display 506 may be a panel that includes light-emitting diodes (LEDs), organic LEDs, liquid crystal elements, or electrophoretic elements. In some embodiments, the display 506 is touch sensitive. Thus, a user may be able to provide input to the diagnostic platform 510 by interacting with the display 506 instead of, or in addition to, interacting with another control mechanism.

The communication module 508 may be responsible for managing communications between the components of the computing device 500, or the communication module 508 may be responsible for managing communications with other computing devices (e.g., server system 408 of FIG. 4). The communication module 508 may be wireless communication circuitry that is designed to establish communication channels with other computing devices. Examples of wireless communication circuitry include chips configured for Bluetooth, Wi-Fi, NFC, and the like.

The nature, number, and type of communication channels established by the communication module 508 may depend on the sources from which information is acquired by the diagnostic platform 510 and the destinations to which information is sent by the diagnostic platform 510. Assume, for example, that the diagnostic platform 510 resides on a computer server of a network-accessible server system. In such embodiments, the communication module 508 may communicate with an imaging device that is responsible for generating digital images of patients. Moreover, the communication module 508 may communicate with a computer program executing on a computing device, such as a mobile phone, desktop computer, or mobile workstation. The computing device could be associated with a healthcare professional who can serve as a grader of the digital images generated by the imaging device following analysis by the diagnostic platform 510.

For convenience, the diagnostic platform 510 may be referred to as a computer program that resides within the memory 504. However, the diagnostic platform 510 could be comprised of software, firmware, or hardware components that are implemented in, or accessible to, the computing device 500. As shown in FIG. 5, the diagnostic platform 510 may include a processing module 512, a diagnostic module 514, an analysis module 516, and a notification module 518. These modules can be an integral part of the diagnostic platform 510. Alternatively, these modules can be logically separate from the diagnostic platform 510 but operate “alongside” it. Together, these modules may enable the diagnostic platform 510 to apply at least one diagnostic models to digital images acquired as input and then determine, based on the outputs produced by those diagnostic models, whether any digital images warrant further examination.

The processing module 512 may be responsible for applying operations to the pixel data of digital images acquired by the diagnostic platform 510. For example, the processing module 512 may process (e.g., denoise, filter, or otherwise alter) the pixel data so that it is usable by the other modules of the diagnostic platform 510. In some embodiments, the diagnostic platform 510 is configured to acquire raw digital images generated by an imaging device. In other embodiments, the diagnostic platform 510 is configured to acquire Digital Imaging and Communications in Medicine (DICOM) data objects, each of which includes pixel data corresponding to a digital image and context data related to attributes of the digital image. In such embodiments, the processing module 512 may be responsible for extracting the pixel data from each DICOM data object for analysis by the other modules. The context data may include information regarding the patient whose body is captured in the digital image, the imaging device responsible for generating the digital image, or the digital image itself.

After pixel data corresponding to a digital image is acquired, the diagnostic module 514 can identify an appropriate diagnostic model to apply to the pixel data. Generally, the diagnostic model is one of multiple diagnostic models maintained in a data structure stored in the memory 504. Each diagnostic model may be associated with a different type of pathological condition. For example, the data structure may include separate diagnostic models for diabetic retinopathy, AMD, glaucoma, and the like. In some embodiments, the data structure also includes diagnostic models that are associated with different types of imaging devices. For example, the data structure may include a first diagnostic model that is trained to identify evidence of diabetic retinopathy using digital images generated by a first brand of retinal camera, a second diagnostic model that is trained to identify evidence of diabetic retinopathy using digital images generated by a second brand of retinal camera, etc. Each diagnostic model can be comprised of one or more algorithms that, when applied to the pixel data of a digital image, produce an output that indicates the health state of the corresponding patient.

The analysis module 516 may be responsible for determining what actions, if any, are appropriate based on the output(s) produced by the diagnostic model(s) applied by the diagnostic module 514. For example, a diagnostic model produces an output indicative of a positive diagnosis for a pathological condition, the analysis module 516 may store data related to the positive diagnosis in a structure associated with the corresponding patient. This structure may be formatted in accordance with a medical image standard. Meanwhile, this data may be related to the corresponding patient, positive diagnosis, diagnosis session, imaging device, etc. For example, the analysis module 516 may indicate, in the record, characteristics (e.g., size, location, classification) of features determined by the diagnostic model to be evidence of the pathological condition.

The notification module 518 may be responsible for notifying one or more graders in response to a determination that further examination of a digital image is warranted based on the output(s) produced by the digital model(s) applied by the diagnostic module 514. Generally, upon discovering that a patient may have a pathological condition, the notification module 518 transmits a notification to a list of graders in an attempt to provoke further examination of the corresponding digital image. At a high level, the notification may be representative of an effort to have at least one of the graders confirm or reject the diagnosis of the patient that was determined by a diagnostic model. The notification may be in the form of a text message or email message that is delivered to a phone number or email address associated with each grader, or the notification may be in the form of a push notification that is generated by a computer program (e.g., a mobile application) executing on a computing device (e.g., a mobile phone) associated with each grader.

As further discussed below, the notification module 518 may also cause transmission of notifications in other situations. For example, the notification module 518 could generate a notification in response to a determination that quality of a digital image falls below a threshold. In such a scenario, the notification could be delivered to the individual responsible for overseeing the diagnosis session in which the digital image was generated, or the notification could be delivered to grader(s) who are responsible for indicating whether quality is sufficient for grading purposes.

Other modules could also be included as part of the diagnostic platform 510. For instance, a graphical user interface (GUI) module 520 may be responsible for generating the interfaces through which individuals can interact with the diagnostic platform 510, view outputs produced by the aforementioned diagnostic modules, receive notifications, perform reads of digital images, etc. As an example, a visualization that is representative of a notification and includes a digital image to which a diagnostic model has been applied may be posted to an interface shown on the display 506 by the GUI module 520 for examination by a grader.

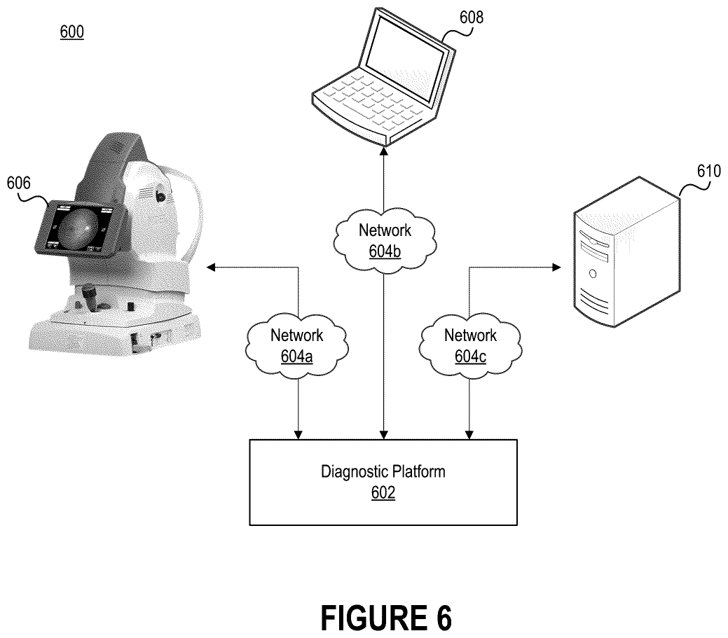

FIG. 6 depicts an example of a communication environment 600 that includes a diagnostic platform 602 configured to acquire data from one or more sources. Here, the diagnostic platform 602 may receive data from a retinal camera 606, laptop computer 608, or network-accessible server system 610 (collectively referred to as the “networked devices”). For example, the diagnostic platform 602 may obtain pixel data from the retinal camera 606, a diagnostic model from the network-accessible server system 610, and input indicative of a confirmation or rejection of an output produced by the diagnostic model upon being applied to the pixel data from the laptop computer 608. Note that the diagnostic platform 602 can, and often will, obtain pixel data that is representative of digital images from more than one retinal camera. For example, the diagnostic platform 602 may obtain pixel data from retinal cameras located in different geographical locations (e.g., in different healthcare facilities).

The networked devices can be connected to the diagnostic platform 602 via one or more networks 604a-c. The networks 604a-c can include PANS, LANS, WANS, MANs, cellular networks, the Internet, etc. Additionally or alternatively, the networked devices may communicate with one another over a short-range wireless connectivity technology, such as Bluetooth or NFC. For example, if the diagnostic platform 602 resides on the network-accessible server system 610, data received from the network-accessible server system 610 need not traverse any networks. However, the network-accessible server system 610 may be connected to the retinal camera 606 and laptop computer 608 via separate Wi-Fi communication channels.

Embodiments of the communication environment 600 may include a subset of the networked devices. For example, some embodiments of the communication environment 600 include a diagnostic platform 602 that receives pixel data from the retinal camera 606 (e.g., in the form of DICOM data objects) and additional data from the network-accessible server system 610 on which it resides. As another example, some embodiments of the communication environment 600 include a diagnostic platform 602 that receives pixel data from a series of retinal cameras located in different environments (e.g., different clinics).

### Methodologies for Autonomously Screening Patients

The prevalence of imaging devices—like retinal cameras—have made it easier than ever before to image anatomical regions for the purpose of diagnosing pathological conditions. Queuing digital images for examination by appropriate healthcare professionals is not a trivial task, however. In fact, delays of several hours to several days are not unusual, even though the speed of referral for treatment can make a major difference in the efficacy of treatment. Introduced here are approaches to assessing digital images generated during image capture sessions using diagnostic models and then stratify patients for examination based on the outputs produced by those diagnostic models.

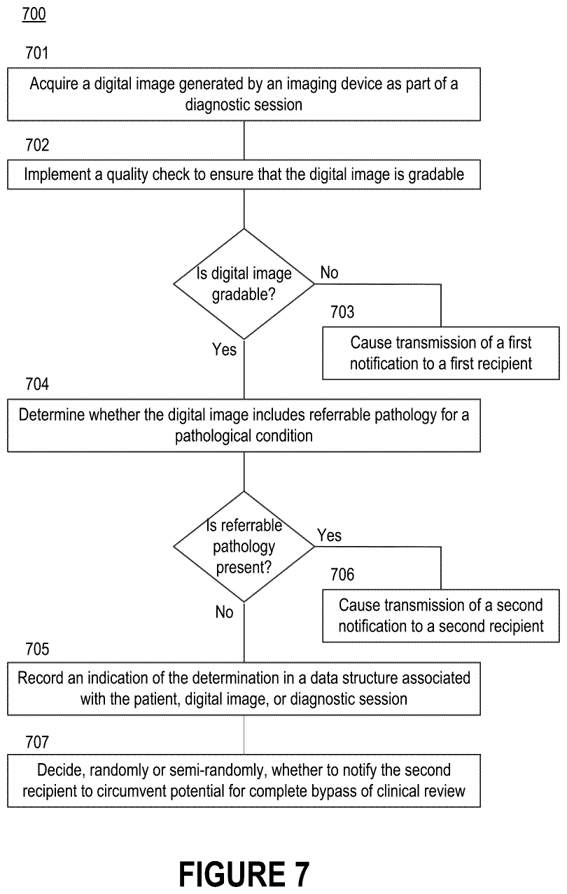

FIG. 7 includes a flow diagram of a process 700 for determining whether a digital image associated with a patient warrants further examination. Initially, a diagnostic platform can acquire a digital image generated by an imaging device (step 701). Generally, the digital image is generated by the imaging device as part of a diagnostic session. Multiple digital images may be generated over the course of the diagnostic session, and each of these digital images may be analyzed by the diagnostic platform. Accordingly, the process 700 could be performed for some or all of the digital images that are generated as part of the diagnostic session.

In some embodiments, the digital image is received from the imaging device in near real time as generation occurs. In such embodiments, the imaging device may be configured to stream digital images to the diagnostic platform as those digital images are generated. In other embodiments, the digital image is received from the imaging device as part of a set. For example, the imaging device may upload all of the digital images generated during a diagnostic session to the diagnostic platform at the conclusion of the diagnostic session. As another example, the imaging device may upload all of the digital images generated over an interval of time (e.g., 4 hours, 8 hours, or 24 hours) to the diagnostic platform at the conclusion of the interval of time. Digital images could also be uploaded in near real time, for example, seconds or minutes after generation by the imaging device. Accordingly, digital images may be continually or periodically acquired by the diagnostic platform for analysis.

In some embodiments, the diagnostic platform implements a quality check (step 702) in response to receiving the digital image. For example, in response to acquiring the digital image, the diagnostic platform may determine whether quality of the digital image is sufficient so as to be gradable through visual analysis. To determine whether the digital image is gradable, the diagnostic platform may implement rules, heuristics, or algorithms that consider characteristics such as signal-to-noise ratio, blurriness, contrast, vignetting, field of view, and the like. As an example, the diagnostic platform may implement an algorithm that computes, infers, or otherwise determines the blurriness of the digital image and then compares the blurriness to a threshold. If the digital image is found to be gradable (e.g., blurriness does not exceed the threshold), then the diagnostic platform may continue with the process 700.

However, if the digital image is found to not be gradable (e.g., blurriness exceeds the threshold), then the diagnostic platform may cause transmission of a first notification to a first recipient (step 703). The first recipient could be the person who is responsible for operating the imaging device, in which case the first notification may prompt the first recipient to generate another digital image. Alternatively, the first recipient could be a grader, in which case the first notification may prompt the first recipient to confirm whether quality of the digital image was properly assessed by the diagnostic platform. Note that the term “grader,” as used herein, could be used to refer to a human or a computer program (e.g., comprising one or more algorithms) that is designed, trained, and implemented to mimic the analysis that has historically been performed by human graders. In embodiments where the grader is a computer program, the computer program generally will execute algorithms that rely on machine learning and/or artificial intelligence to better understand the content and context of the digital images. The grader may be able to provide insight or context regarding the poor quality. For example, a non-trivial percentage of the population cannot have their retinas imaged without dilation. The grader may be able to determine, based on an examination of the digital image, whether the patient should be reimaged following dilation. This determination may be based on an inability to clearly observe physiological structures inside the eye. If the grader indicates that quality of the digital image is poor yet fixable, then the diagnostic platform may cause transmission of another notification to the operator in an attempt to provoke capture of another digital image.

Then, the diagnostic platform can determine whether the digital image includes referable pathology for a pathological condition (step 704). For example, the diagnostic platform may obtain a diagnostic model that has been trained to identify evidence of the pathological condition, apply the diagnostic model to the digital image, and then determine whether the digital image includes referable pathology based on the output produced by the diagnostic model. In embodiments where the output specifies a proposed diagnosis, the diagnostic platform may determine that the digital image includes referable pathology in response to a determination that the proposed diagnosis is a positive diagnosis for the pathological condition. In embodiments where the output specifies evidence of the pathological condition (e.g., in the form of bounding boxes identifying regions of pixels corresponding to the evidence), the diagnostic platform may determine that the digital image includes referable pathology in response to a determination that the proposed diagnosis includes at least a predetermined amount of evidence of the pathological condition.

If the diagnostic platform determines that the digital image does not include referable pathology for the pathological condition, then the diagnostic platform may simply record an indication of the determination in a data structure (step 705) that is associated with the patient, digital image, diagnostic session, or any combination thereof. The data structure may be representative of a health record that is maintained over time for the patient. At a high level, the diagnostic platform may determine that the digital image does not include referable pathology if (i) there is no quantifiable evidence of the pathological condition or (ii) the amount of quantifiable evidence of the pathological condition does not exceed a predetermined threshold. The amount of quantifiable evidence may also correspond to confidence in the prediction regarding the pathological condition. Accordingly, the diagnostic platform may determine that the digital image does not include referable pathology if only small amounts of quantifiable evidence are discovered. Moreover, the diagnostic platform may determine that the digital image does not include referable pathology if it determines there is a low likelihood (e.g., less than 10, 20, 30, or 50 percent) of the pathological condition, or the diagnostic platform may determine that the digital image does not include referable pathology if confidence in its prediction of an affirmative diagnosis of the pathological condition falls beneath a threshold (e.g., 50, 40, or 30 percent).

However, if the diagnostic platform determines that the digital image includes referable pathology for the pathological condition, then the diagnostic platform may cause transmission of a second notification to a second recipient (step 706). Generally, the second recipient is a grader who is responsible for examining the digital image to determine whether treatment of the patient for the pathological condition is warranted. Thus, transmission of the second notification may be intended to prompt the second recipient to examine the digital image in a timely manner (e.g., within several minutes of the diagnostic session). The second recipient could be the same person as the first recipient in some situations, though that need not necessarily be the case. For example, the first recipient could be the operator of the imaging device, another grader, or a person who specializes in examining digital images for quality determination purposes rather than for diagnostic purposes.

While embodiments may be described in the context of determining the quality of a digital image through analysis of its content, the diagnostic platform could additionally or alternatively consider other factors. For example, the diagnostic platform may consider procedural criteria that indicate how the digital image was generated, how the digital image was routed from the imaging device to the diagnostic platform, and the like. As another example, the diagnostic platform could consider metadata criteria. Specifically, the diagnostic platform could determine quality of the digital image through analysis of its metadata, for example, to determine the provenance of the digital image, integrity of the source from which the digital image is obtained, consistency of the metadata, expiration date, or whether the metadata supports information related to the diagnostic session, for example, indicating who the patient is, where the imaging device is located, what type of imaging device was used, when the diagnostic session occurred, why the diagnostic session was scheduled, and the like.

Note that, in some embodiments, the decision whether to cause transmission of the second notification may have some degree of randomness. As discussed above, if the diagnostic platform determines that the digital image includes evidence of the pathological condition, then the second recipient can be notified of the determination. However, there will be situations where the diagnostic platform determines that the digital image does not include any evidence of the pathological condition. For example, when applied to the digital image, the diagnostic model may produce an output that is representative of a negative diagnosis for the pathological condition. In such situations, the diagnostic platform may simply record an indication of the determination in a data record as noted above. Additionally or alternatively, the diagnostic platform may randomly or semi-randomly decide whether to notify the second recipient in an effort to circumvent the potential for independent action to completely bypass clinical review by a healthcare professional (step 707). The rate of randomness can be adjusted based on, for example, the availability of the second recipient, the desired level of “backup” review, and the like. Accordingly, the diagnostic platform may prompt the second recipient to review the digital image even if the diagnostic platform determines no evidence of the pathological condition exists, as a means of ensuring that live clinical review is not bypassed on too broad of a scale.

This further examination by the second recipient can also serve as a check on the number of false negatives that are produced by the diagnostic model. The diagnostic platform may be able to “tune” the diagnostic model based on whether feedback received from the second recipient aligns with the outputs produced by the diagnostic. For example, if the diagnostic platform determines, based on responses to notifications transmitted to the second recipient, that the rate of false negatives exceeds a threshold (e.g., 1 percent, 2 percent, or 5 percent), then the diagnostic platform may initiate retraining of the diagnostic model. Feedback received from the second recipient could also be used to monitor the number of false positives that are produced by the diagnostic model. As mentioned above, the second recipient may be notified to examine the digital image in response to a determination that evidence of the pathological condition is included therein. If the second recipient indicates (e.g., by interacting with the notification) that the patient does not have the pathological condition, then the diagnostic platform may categorize its initial determination as a false positive. Similar to false negatives, the diagnostic platform may initiate retraining of the diagnostic model if the rate of false positives exceeds a threshold (e.g., 1 percent, 2 percent, or 5 percent). The threshold for false negatives does not need to be identical to the threshold for false positives.

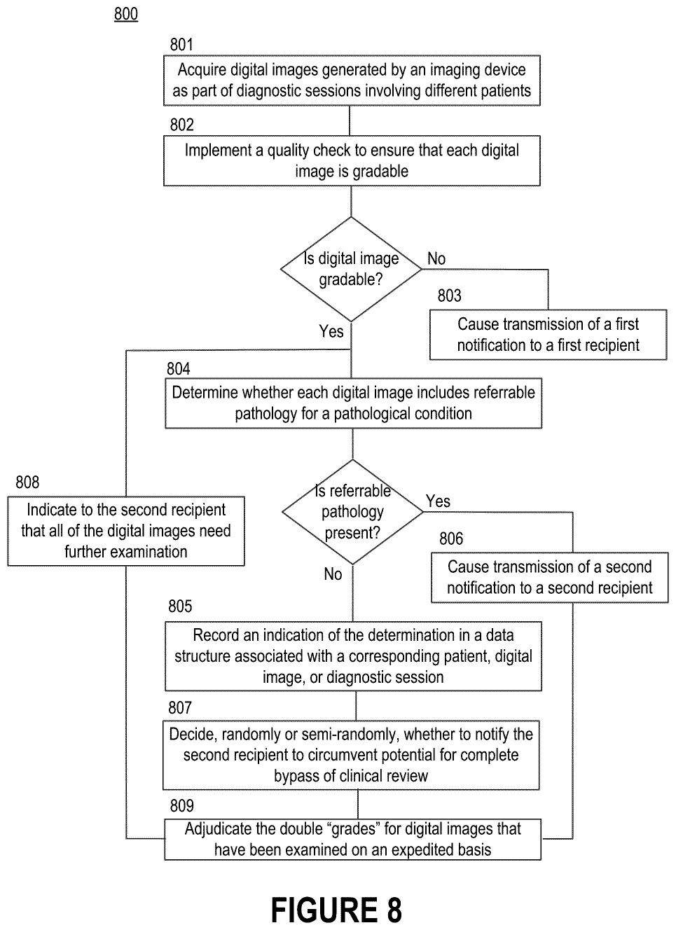

FIG. 8 includes a flow diagram of a process 800 for determining whether digital images associated with different patients warrant further examination. Steps 801-807 of FIG. 8 may be comparable to steps 701-707 of FIG. 7. Notably, these steps are performed for multiple digital images in FIG. 8. This will result in a subset of the digital images-namely, all of those digital images determined to include evidence of the pathological condition and some of those digital images determined to not include evidence of the pathological condition-being further examined in a timely manner.

Here, however, the diagnostic platform further sends all of the digital images-including the ones identifies for expedited examination-through the grading process. More specifically, the diagnostic platform can indicate to the second recipient that all of the digital images need further examination (step 808). While the second recipient may be notified that all of the digital images need further examination, this does not need to happen simultaneously. For example, the second recipient may be notified over time as the digital images are acquired by the diagnostic platform.

As mentioned above, the second recipient is normally one of multiple recipients who are notified of the digital images that require examination, expedited and otherwise. Accordingly, while the second recipient may be notified that all of the digital images need further examination, the second recipient may not actually examine all of the digital images. Instead, the digital images could be examined by a pool of graders, each of whom may be notified when a digital image is ready for examination.

As feedback is obtained from the second recipient, the diagnostic platform can catalogue the “grade” that is assigned to each digital image. Each “grade” may be representative of a proposed diagnosis that is determined by the second recipient based on an analysis of the corresponding digital image. Some of the digital images will be graded twice, namely, once on an expedited basis and once on a normal basis. The diagnostic platform can adjudicate the double grades for digital images that have been examined on an expedited basis (step 809). For example, the diagnostic platform may indicate in the data structure whether the grades agree with one another, the degree to which the grades disagree, which grade is considered correct, etc.

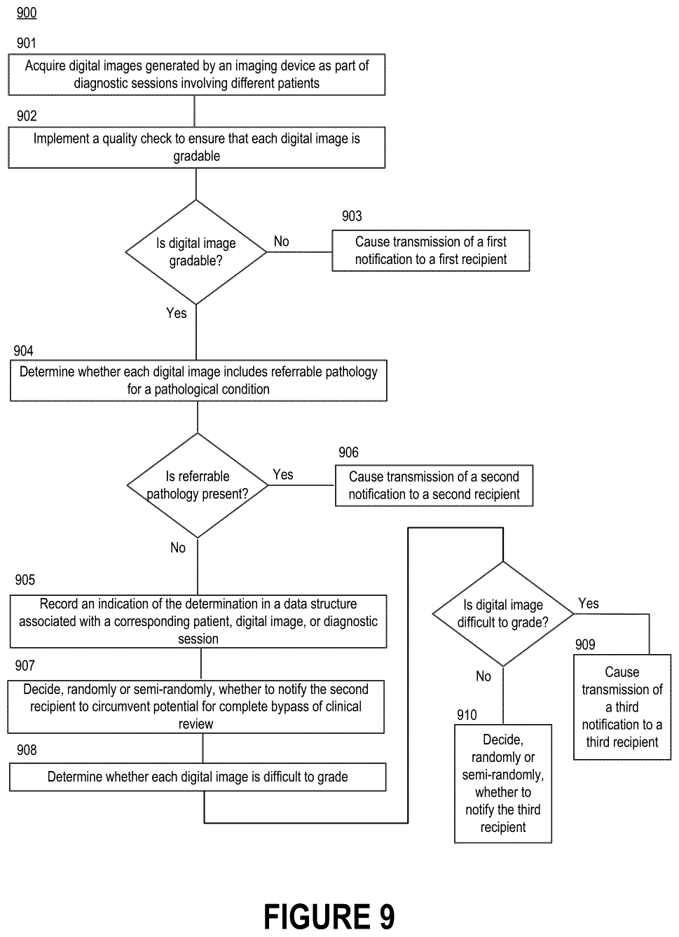

FIG. 9 includes a flow diagram of a process 900 for handling digital images that are difficult to grade. Steps 901-907 of FIG. 9 may be comparable to steps 701-707 of FIG. 7. Like FIG. 8, these steps are performed for multiple digital images in FIG. 9. This will result in a subset of the digital images—namely, all of those digital images determined to include evidence of the pathological condition and some of those digital images determined to not include evidence of the pathological condition-being further examined in a timely manner.

As shown in FIG. 9, the diagnostic platform can also determine whether each digital image is difficult to grade (step 908). Grading difficulty could also be established based on the amount of confidence in the output that is produced by the diagnostic model. Assume, for example, that the diagnostic model is designed to produce, as output, weights for different classifications of diabetic retinopathy. In such a scenario, whether a given digital image is considered difficult to grade may be based on whether there is multimodal distribution of weights or perturbation (e.g., in color space) when the diagnostic model is applied thereto. Said another way, if the diagnostic model cannot clearly identify one classification of diabetic retinopathy, then the diagnostic platform may deem the given digital image to be difficult to grade. A similar approach may be taken if the diagnostic model is designed to produce, as output, weights for positive and negative diagnoses of a pathological condition (e.g., glaucoma). If neither of the weights exceeds a threshold (e.g., 70 percent, 80 percent, or 90 percent) when the diagnostic model is applied to a given digital image, then the diagnostic platform may deem the given digital image to be difficult to grade.

If the diagnostic platform determines that a given digital image is difficult to grade, then the diagnostic platform may cause transmission of a third notification to a third recipient (step 909). Generally, the second and third recipients are both graders who are able to determine whether treatment for a pathological condition is warranted through visual analysis of digital images. However, the third recipient may be more skilled (e.g., have more experience) than the second recipient, and therefore better suited for examining digital images that are difficult to grade. If the diagnostic platform determines that the given digital image is not difficult to grade, then the diagnostic platform may not necessarily notify the third recipient. Instead, the diagnostic platform may decide, randomly or semi-randomly, whether to notify the third recipient (step 910). This can be done for load management purposes, as well as to ensure that the diagnostic platform is properly identifying digital images that are difficult to grade. Adjudication can be performed, as necessary, for digital images that have been assigned more than one grade as discussed above with reference to FIG. 8.

In some embodiments, the diagnostic platform may also decide, randomly or semi-randomly, whether to send digital images with referable pathology to a grader (e.g., the first, second, or third recipient). For example, the diagnostic platform may randomly decide to send digital images with referable pathology to graders that are assigned fewer digital images with referable pathology. This can be done for quality assurance purposes, as well as to ensure that graders remain engaged. Again, adjudication can be performed, as necessary, for digital images that have been assigned more than one grade as discussed above with reference to FIG. 8.

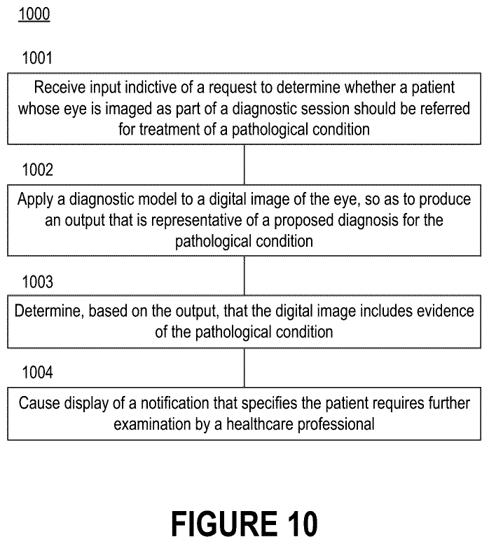

FIG. 10 depicts a flow diagram of a process 1000 for establishing whether expedited examination of a digital image associated with a patient is warranted. Initially, a diagnostic platform can receive input indicative of a request to determine whether a patient whose eye is imaged as part of a diagnostic session should be referred for treatment of a pathological condition (step 1001). In some embodiments, the input is representative of receipt of a digital image from a source. The source could be the imaging device that generates the digital image, or the source could be a storage medium that is accessible to the diagnostic platform.

The diagnostic platform can then apply a diagnostic model to the digital image of the eye, so as to produce an output that is representative of a proposed diagnosis for the pathological condition (step 1002). In some embodiments, the diagnostic model is selected by the diagnostic platform from among multiple diagnostic models that are stored in a data structure. Each of the multiple diagnostic models may be trained to identify evidence of a different pathological condition through analysis of pixel information.

Thereafter, the diagnostic platform may determine, based on the output, that the digital image includes evidence of the pathological condition (step 1003). For example, the diagnostic platform may determine that the digital image includes evidence of the pathological condition in response to a determination that the output is representative of a positive diagnosis for the pathological condition. As another example, the diagnostic platform may determine that the digital image includes evidence of the pathological condition in response to a determination that the output identifies one or more contiguous regions of pixels representative of pathology for the pathological condition.

Then, the diagnostic platform can cause display of a notification that specifies the patient requires further examination by a healthcare professional (step 1004). In some embodiments, the notification is displayed to the patient, so as to prompt the patient to seek further examination by the healthcare professional. For example, the notification may specify that the patient should schedule a physical examination with the healthcare professional due to evidence of the pathological condition. In other embodiments, the notification is displayed to the healthcare professional who is responsible for rendering an actual diagnosis based on an analysis of the digital image. In such embodiments, the healthcare professional may be one of multiple healthcare professionals that are notified that the digital image requires further examination. For example, the diagnostic platform may cause transmission of a notification to a list of healthcare professionals, any of whom may be permitted to examine the digital image to provide the actual diagnosis. Alternatively, the notification may be transmitted in a more tailored manner. For example, the diagnostic platform may select the healthcare professional to further examine the digital image from among a pool of healthcare professionals. Assume, for example, that the diagnostic platform (i) applies multiple models associated with different pathological conditions to the digital image and (ii) discovers that the output produced by one of the multiple models is representative of a positive diagnosis. In such a scenario, the healthcare professional may be identified based on the positive diagnosis. For example, the healthcare professional may be considered an expert in diagnosing the pathological condition for which a positive diagnosis was proposed. Accordingly, the diagnostic platform may iterate through multiple models corresponding to different pathological conditions to determine a set of outputs indicative of a set of next step diagnoses corresponding to the different pathological conditions. For each pathological condition, the corresponding diagnosis may indicate, to the diagnostic platform, an appropriate next step.

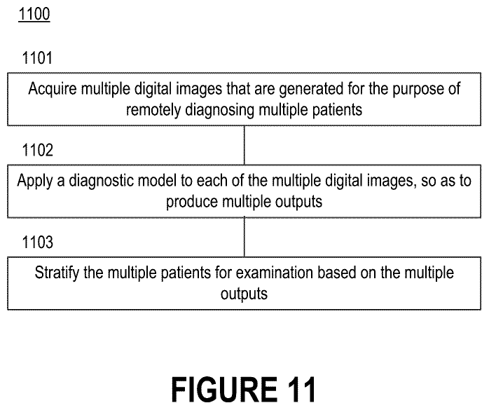

FIG. 11 depicts a flow diagram of a process 1100 for stratifying patients whose retinas have been imaged for further examination. While the process 1100 is described in the context of ocular diseases, those skilled in the art will recognize that the process 1100 may be similarly applicable to other pathological conditions.

Initially, a diagnostic platform can acquire multiple digital images that are generated for the purpose of remotely diagnosing multiple patients (step 1101). For each patient, the diagnostic platform may acquire at least one digital image that is generated as part of a diagnostic session in which the eye is imaged.

Then, the diagnostic platform can apply a diagnostic model to each of the multiple digital images, so as to produce multiple outputs (step 1102). Each output may indicate whether pathological features that are indicative of an ocular disease (e.g., diabetic retinopathy) are present in the corresponding digital image. Several examples of diagnostic models are provided below:
Binary Classification Models: The output of a binary classification model specifies one of two classes. An example of such a model is one that when applied to a digital image, determines whether to positively or negatively diagnose the corresponding patient with respect to a pathological condition.
Non-Binary Classification Models: The output of a non-binary classification model specifies one of at least three classes. An example of such a model is one that when applied to a digital image, specifies whether the corresponding patient has no evidence of diabetic retinopathy, evidence of mild diabetic retinopathy, evidence of moderate diabetic retinopathy, or evidence of severe diabetic retinopathy.
Regression Models: The output of a regression model is a single number (e.g., 50 percent) or an interval of numbers (e.g., 40-60 percent). An example of such a model is one that when applied to a digital image, estimates the probability that the corresponding patient has a pathological condition.

Thereafter, the diagnostic platform can stratify the multiple patients f based on the multiple outputs (step 1103). Generally, the multiple patients are stratified for examination purposes based on the multiple outputs. For example, the diagnostic platform may produce, based on the multiple outputs, a ranked list of the multiple patients, such that patients who are determined to exhibit more evidence of the ocular disease are ranked higher than individuals who are determined to exhibit less evidence of the ocular disease. The diagnostic platform can then cause the multiple patients to be presented to at least one healthcare professional, either simultaneously or sequentially, for examination in order of the ranked list. As another example, the diagnostic platform may produce, based on the multiple outputs, at least two lists, each of which may include a subset of the multiple patients. Each list may be associated with a different type of healthcare professional. For example, the diagnostic platform may produce one list of patients to be further examined by a primary care physician, a second list of patients to be further examined by an optometrist, a third list of patients to be further examined by an ophthalmologist, etc. Generally, the different types of healthcare professionals (and therefore, the lists) depend on the nature of the diagnostic models that are to be applied by the diagnostic platform. Said another way, how patients are stratified among different healthcare professional may depend on the pathological conditions for which evidence is sought.

Additionally or alternatively, the multiple patients may be stratified based on the quality of the corresponding digital images. For example, the diagnostic platform may assign, based on the multiple outputs or its analysis of the multiple digital images, the multiple patients among three categories. The first category may include those patients that require additional digital images. Patients may be assigned to the first category if the quality of the corresponding digital images are determined to be too low (e.g., and therefore ungradable by the diagnostic model), or patients may be assigned to the first category if confidence in the output produced by the diagnostic model upon being applied to the corresponding digital images is low (e.g., falls below a threshold). The second category may include those patients for which no further examination is needed. Patients may be assigned to the second category if the outputs produced by the diagnostic model upon being applied to the corresponding digital images indicate that there is little or no evidence of the pathological condition. Thus, patients assigned to the second category may be associated with negative diagnoses output by the diagnostic model. The third category may include those patients for which further examination is needed. Patients may be assigned to the third category if the outputs produced by the diagnostic model upon being applied to the corresponding digital images indicate that there is evidence of the pathological condition. Thus, patients assigned to the third category may be associated with positive diagnoses output by the diagnostic model. For those patients in the third category, the diagnostic platform may generate visualizations that are intended to assist the healthcare professional(s) in rendering actual diagnoses. For each patient included in the third category, the diagnostic platform may automatically generate content-like comments and feedback-regarding the corresponding digital image. For example, the diagnostic platform may highlight areas of the digital image in an attempt to alert the healthcare professional to a certain feature (e.g., that caused the diagnostic model to output a positive diagnosis). As another example, the diagnostic platform may identify a data discrepancy or health metadata associated with the input (e.g., the DICOM data object) that may be diagnostically relevant.

Unless contrary to physical possibility, it is envisioned that the steps described above may be performed in various sequences and combinations. For example, the processes could be performed as digital images are acquired by the diagnostic platform, such that those digital images that warrant further examination are consistently expedited for prompt consideration. As another example, the processes could be performed in near real time as digital images are generated. Said another way, digital images may be examined by the diagnostic platform upon acquisition to ensure that patients are promptly queued for examination purposes based on presence or severity of the pathological condition.

Other steps may also be included in some embodiments. For example, the model may be one of multiple models that are applied to the digital image of the eye by the diagnostic platform, so as to produce multiple outputs, each of which may be representative of a proposed diagnosis for a different pathological condition. In embodiments where the diagnostic platform applies multiple models to each digital image, the diagnostic platform may decide whether each patient warrants further examination based on an analysis (e.g., a weighted analysis) of the multiple outputs.

### Processing System

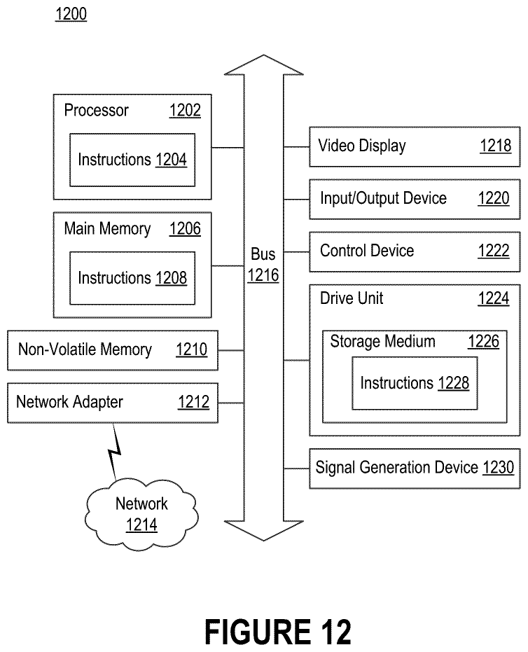

FIG. 12 is a block diagram illustrating an example of a processing system 1200 that can perform at least some operations described herein. For example, some components of the processing system 1200 may be hosted on a computing device that includes a diagnostic platform (e.g., diagnostic platform 402 of FIG. 4 or diagnostic platform 510 of FIG. 5).

The processing system 1200 may include a processor 1202, main memory 1206, non-volatile memory 1210, network adapter 1212, video display 1218, input/output device 1220, control device 1222 (e.g., a keyboard or pointing device), drive unit 1224 including a storage medium 1226, and signal generation device 1230 that are communicatively connected to a bus 1216. The bus 1216 is illustrated as an abstraction that represents one or more physical buses or point-to-point connections that are connected by appropriate bridges, adapters, or controllers. The bus 1216, therefore, can include a system bus, a Peripheral Component Interconnect (PCI) bus or PCI-Express bus, a HyperTransport or industry standard architecture (ISA) bus, a small computer system interface (SCSI) bus, a universal serial bus (USB), inter-integrated circuit (I2C) bus, or an Institute of Electrical and Electronics Engineers (IEEE) standard 1394 bus (also referred to as “Firewire”).

While the main memory 1206, non-volatile memory 1210, and storage medium 1226 are shown to be a single medium, the terms “machine-readable medium” and “storage medium” should be taken to include a single medium or multiple media (e.g., a centralized/distributed database and/or associated caches and servers) that store one or more sets of instructions 1228. The terms “machine-readable medium” and “storage medium” shall also be taken to include any medium that is capable of storing, encoding, or carrying a set of instructions for execution by the processing system 1200.

In general, the routines executed to implement the embodiments of the disclosure may be implemented as part of an operating system or a specific application, component, program, object, module, or sequence of instructions (collectively referred to as “computer programs”). The computer programs typically comprise one or more instructions (e.g., instructions 1204, 1208, 1228) set at various times in various memory and storage devices in a computing device. When read and executed by the processors 1202, the instruction(s) cause the processing system 1200 to perform operations to execute elements involving the various aspects of the present disclosure.

Further examples of machine- and computer-readable media include recordable-type media, such as volatile memory and non-volatile memory 1210, removable disks, hard disk drives, and optical disks (e.g., Compact Disk Read-Only Memory (CD-ROMS) and Digital Versatile Disks (DVDs)), and transmission-type media, such as digital and analog communication links.

The network adapter 1212 enables the processing system 1200 to mediate data in a network 1214 with an entity that is external to the processing system 1200 through any communication protocol supported by the processing system 1200 and the external entity. The network adapter 1212 can include a network adaptor card, a wireless network interface card, a router, an access point, a wireless router, a switch, a multilayer switch, a protocol converter, a gateway, a bridge, bridge router, a hub, a digital media receiver, a repeater, or any combination thereof.

### Remarks

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling those skilled in the relevant art to understand the claimed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments may vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

What is claimed is:

1. A non-transitory medium with instructions stored thereon that, when executed by a processor of a computing device, cause the computing device to perform operations comprising:
receiving input indicative of a request to determine whether an individual whose eye is imaged as part of a diagnostic session should be referred for treatment of a pathological condition;
applying a model to a digital image of the eye, so as to produce an output that is associated with a proposed next step diagnosis for the pathological condition;
determining, based on the output, that the digital image includes evidence of the pathological condition; and
causing display of a notification that specifies the individual requires the proposed next step diagnosis by a healthcare professional.

2. The non-transitory medium of claim 1, wherein the operations further comprise:
selecting the model from among multiple models stored in a data structure,
wherein each model of the multiple models is trained to identify evidence of a different pathological condition through analysis of pixel information.

3. The non-transitory medium of claim 1, wherein the operations further comprise:
iterating through multiple models corresponding to different pathological conditions to determine a set of outputs indicative of a set of next step diagnoses corresponding to the different pathological conditions;
wherein the model is one of multiple models applied to the digital image of the eye, such that multiple outputs are produced, and
wherein each output of the multiple outputs is indicative of a proposed diagnosis for a different pathological condition.

4. The non-transitory medium of claim 3, wherein the operations further comprise:
configuring the notification based on which of the multiple outputs indicate presence of a corresponding pathological condition,
wherein said configuring includes selecting the healthcare professional from among multiple healthcare professionals or assigning multiple pathological conditions to the healthcare professional.

5. The non-transitory medium of claim 1, wherein the notification is displayed to the healthcare professional who is responsible for rendering an actual diagnosis corresponding to the proposed next step diagnosis.

6. The non-transitory medium of claim 1,
wherein the input is representative of receipt of the digital image from a source, and
wherein said applying is performed in response to said receiving, said determining is performed in response to said applying, and said causing is performed in response to said determining, such that the notification is produced in near real time with the generation of the digital image.

7. The non-transitory medium of claim 1,
wherein the digital image is one of multiple digital images that are generated by an imaging device during the diagnostic session, and
wherein the multiple images are received from the imaging device following a conclusion of the diagnostic session.

8. The non-transitory medium of claim 1,
wherein the input is representative of receipt of the digital image from a source,
wherein the operations further comprise:
establishing that quality of the digital image is sufficient so as to be gradable through visual analysis, and
wherein said applying is performed in response to said establishing.

9. The non-transitory medium of claim 8, wherein said establishing involves the implementation of a rule, heuristic, or algorithm that considers how signal-to-noise ratio, blurriness, contrast, vignetting, or field of view compares to a threshold.

10. A method comprising:
acquiring, by a processor, multiple digital images that are generated for the purpose of remotely diagnosing multiple individuals,
wherein each digital image of the multiple digital images is associated with a corresponding individual of multiple individuals;
applying, by the processor, a model to the multiple digital images, so as to produce multiple outputs,
wherein each output of the multiple outputs is associated with a proposed next step diagnosis for a pathological condition for the corresponding individual; and
stratifying, by the processor, the multiple individuals based on the multiple outputs.

11. The method of claim 10, wherein said stratifying comprises:
assigning the multiple individuals among a first category, a second category, and a third category,
wherein the first category includes those individuals, if any, for which an additional digital image of higher quality is needed,
wherein the second category includes those individuals, if any, for which the corresponding outputs are representative of negative diagnoses, and
wherein the third category includes those individuals, if any, for which the corresponding outputs are representative of positive diagnoses.

12. The method of claim 10, wherein said stratifying comprises:
producing, based on the multiple outputs, (i) a first list that includes a first subset of the multiple individuals and (ii) a second list that includes a second subset of the multiple individuals,
wherein the first list is associated with a first type of healthcare professional, and
wherein the second list is associated with a second type of healthcare professional.

13. The method of claim 10, wherein said stratifying comprises:
producing, based on the multiple outputs, a ranked list of the multiple individuals, such that individuals who are determined to exhibit more evidence of the pathological condition are ranked higher than individuals who are determined to exhibit less evidence of the pathological condition.

14. The method of claim 13, further comprising:
causing, by the processor, presentation of the multiple individuals to at least one healthcare professional for further examination in order of the ranked list.

15. The method of claim 10, wherein said acquiring, said applying, and said stratifying are performed in near real time with the generation of the multiple digital images, such that the multiple individuals are promptly ranked for examination purposes based on severity of the pathological condition.

16. A method comprising:
acquiring a digital image that is generated as part of a diagnostic session in which an eye of an individual is imaged;
applying a model to the digital image to produce an output that indicates whether pathological features that are indicative of diabetic retinopathy are present in the digital image;
determining, based on the output, that the digital image includes at least one pathological feature that is indicative of diabetic retinopathy; and
causing display of a notification that specifies further examination of the digital image by a healthcare professional is needed.

17. The method of claim 16, wherein the model is a binary classification model that indicates, based on analysis of the digital image, whether the eye is exhibiting evidence of diabetic retinopathy.

18. The method of claim 16, wherein the model is a non-binary model that specifies, based on analysis of the digital image, one of multiple severity classifications to which to assign the individual.

19. The method of claim 16, wherein the model is a regression model that indicates, based on analysis of the digital image, a probability of the individual having diabetic retinopathy.

20. The method of claim 16, further comprising:
generating, based on the output, a visualization component that visually identifies digital features in the digital image that are determined to be representative of the at least one pathological feature by the model;
wherein the visualization component is accessible via the notification.
